(19) United States
(12) Patent Application Publication
(10) Pub. No.: US 2007/0122819 A1
(43) Pub. Date: May 31, 2007

(54) Analyte assay structure in microfluidic chip for quantitative analysis and method for using the same

(75) Inventors: Bi-Chu Wu, Cyonglin Township (TW); Gin-Shu Young, Cyonglin Township (TW)

Correspondence Address: BACON & THOMAS, PLLC, 625 SLATERS LANE, FOURTH FLOOR, ALEXANDRIA, VA, 22314, US

(73) Assignee: Industrial Technology Research Institute, Hsinchu (TW)

(21) Appl. No.: 11/286,405

(22) Filed: Nov. 25, 2005

(51) Int. Cl. C12Q 1/68; C12M 3/00

(52) U.S. Cl. 435/6; 435/287.2

(57) ABSTRACT

The object of the present invention is to provide a sample assay structure in a microfluidic chip for quantitative analysis which comprises a sample inlet port for inputting a testing sample; an analyte detection region, coupled to the sample inlet port, consisting of at least one microfluidic channel, in which a plurality of immobilized substances capable of reacting with the analyte are placed; and a fluid driving device, capable of controlling the speed of the flow of the test sample through the analyte detection region, allowing the quantity of the analyte be indicated by the length of the portion of the microfluidic channel where the analyte reacted with the immobilized substances.

### BACKGROUND OF THE INVENTION

1. Field of the Invention

The present invention relates to a sample assay structure in microfluidic chip for quantitative analysis without the use of an instrument.

2. Description of Related Art

Many applications of clinical biochemical assay focus on the detection of the specific biochemical substances or pathogens that reflect the health or illness of a patient or the effects of medical treatment. The detection of biological and chemical substances, however, can also be applied to screening for drug abuse, to industrial manufacturing processes, to detect environmental pollution, and to the assay of plant and animal samples.

The test sample for the assay is application-dependent. Drug screening or the assay of animal samples may use fluids from human or animals, such as blood, urine, saliva or serum. Industrial manufacturing process or environmental detection can use liquid samples from the manufacturing process and/or the environment. In the present invention the liquid or body fluid used is called the test sample. The various specific components to be detected by the use of the strip or biochip are called analytes. The analyte in the test sample may be a chemical substance, a protein, a ligand, nucleic acid or a pathogenic virus or bacteria.

Depending on the results required of the assay, two types of applications are possible: a qualitative test or a quantitative assay. The qualitative test seeks simply to establish the existence of the analyte. If the analyte is present in an amount above a particular level, either a positive or negative result is obtained. Over the counter pregnancy test strips, for example, seek to determine if the quantity of human chorionic gonadotropin (hCG) in the urine sample is above a certain value. For example, if the hCG is above 25 mIU/ml, the test result from the strip is considered positive, a qualitative result. A quantitative assay determines the specific amount of the analyte in the test sample. In a cholesterol assay, for example, the numeral value obtained will reflect the actual concentration of cholesterol in the blood in (mg/dl).

Bioassays are performed using liquid reagents or using dry strips. When a liquid reagent is used, a large instrument is often needed, for example, the bioanalyzers used for blood and urine tests in large hospitals. Dry strip assays may be performed either alone or with the assistance of a portable instrument. The pregnancy test mentioned above uses a strip providing results that can be read directly from the strip without the use of any instrument. The home glucose assay, on the other hand, is an example of a dry strip test requiring a portable meter to read the results.

Liquid assays are usually limited to hospitals or medical centers because of the bulky size and expense of the instruments, as well as the need for a licensed professional to process the assay. Dry strip assays, on the other hand, are portable and less expensive, thus more appropriate for use in the home or in clinics. Dry strip assays are often qualitative tests because of the difficulty in obtaining accurate readings. To improve the reading accuracy of dry test strips, a portable instrument is often used to obtain quantitative results.

The reasons for the poor reading accuracy of dry strips when used without the use of an instrument can easily be explained. Dry strips are often made of porous fibers to allow the sample liquid to flow from one end of the strip to the other by capillary action. Specific substances immobilized on the reaction zone of the strip react with the analyte as the test sample passes through the pores of the reaction zone. The analyte may be labeled with a color marker. By reading the length or the area of the labeled analyte remaining in the reaction zone after the reaction, the quantity of the analyte can be determined. The larger the colored area, the higher the concentration of the analyte. FIG. 1 (from reference [1]) shows the results of a typical strip after absorbing the liquid sample from the lower end of the test strip. The analyte reacts with the immobilized substance in the reaction zone. The length of the colored zone reflects the concentration of the analyte. Some issues in this approach are observed and discussed:
1. The shape of the reaction zone: The fiber-based strip has to be short enough so the liquid sample can be driven from one end to the other completely by capillary action. However, a shorter strip implies a shorter reaction zone and poor resolution, making it difficult for a user to read the length of the colored zone.
2. Control of the sample flow through the path: When the sample moves upward, the strip has no control over the path of its flow. The liquid may or may not spread evenly along the width of the strip. As shown in FIG. 1, the front edge of the color changed region may have an irregular shape, making it difficult to read the colored length.
3. Speed of the test sample: The quality of the fiber based strip dramatically affects the opportunities for contact and reaction between the analyte and the immobilized substances. A strip with poor fiber uniformity may cause the liquid sample to flow through the reaction zone at varying speeds, causing different reaction patterns. Different gray tones of the labeled analyte in the reaction zone may contribute to inaccuracy in reading the length of the colored area.
4. Control of sample volume: Because of the limitations of the construction of the strip, it is difficult to control precisely the volume of sample flowing through the reaction zone. Accuracy in reading the concentration of the analyte may be dramatically affected.
5. Other functions affecting the assay: Functions such as washing after the reaction, separating blood cells, diluting samples, adding reagents automatically, mixing, assaying multiple analyses, etc., are very useful to enhance the accuracy or capability of an assay. However, based on current construction, it is difficult to attach these functions to dry strips.

To improve the performance of dry strips, some designs replaced the fiber-based strip with a structure with a flat straight space separated by two plates, allowing the sample to flow through the space by capillary action. However, because capillary action is used to drive the sample, the space must be wide and short, causing reading inaccuracies unless an instrument is used.

Besides traditional dry strips for bioassay, biochips, including micro array chips and microfluidic chips, have the potential to be used for in vitro analysis. Micro array chips use an array of spots with immobilized substances in the reaction region. Microfluidic chips often use a reaction chamber in the reaction region. Instruments, including modules for fluorescence excitation or detection, are often required to complete the assay process.

To assay analytes without the use of an instrument, a structure with the ability to provide quantitative results is needed. The ability to integrate pre-and post-processing functions would be a most desirable feature as would the use of a simplified procedure for nonprofessional users.

### SUMMARY OF THE INVENTION

The object of the present invention is to provide a sample assay structure in a microfluidic chip for quantitative analysis which comprises a sample inlet port for inputting a testing sample; an analyte detection region, coupled to the sample inlet port, consisting of at least one microfluidic channel, in which a plurality of immobilized substances capable of reacting with the analyte are placed; and a fluid driving device, capable of controlling the speed of the flow of the test sample through the analyte detection region, allowing the quantity of the analyte be indicated by the length of the portion of the microfluidic channel where the analyte reacted with the immobilized substances.

The reaction beginning point of the present invention means the starting point of placing the immobilized substances in the analyte detection region. The type of immobilized substances used depends on the analyte to be reacted with the immobilized substances. The reaction mechanism may be a chemical reaction or a binding pair reaction. In the case of a binding pair reaction, suitable analyte/immobilized substances pairs may include, but are not limited to, antibodies/antigens, receptors/ligand, proteins/nucleic acids, nucleic acids/nucleic acids, enzymes/substrates and/or inhibitors, carbohydrates (including glycoproteins and glycolipids)/lectins, carbohydrates and other binding partners, proteins/proteins; and protein/small molecules.

The immobilized substances are attached to the analyte detection region before the testing sample enters the analyte detection region. The substances may be attached to the analyte detection region early, during the chip manufacturing process, or later, during the user application process. For example, the immobilized substances attached to magnetic beads may be delivered to the analyte detection region immediately before the analytes are applied to the detection region.

By controlling the speed of the flow of the sample and/or disturbing the sample in the analyte detection region the opportunities for contact between the analyte and the immobilized substances are increased. The immobilized substances in the microfluidic channel sequentially react with the analyte in the sample. When the reaction is finished, immobilized substances that reacted with the analyte concentrate at the front section of microfluidic channel. Those substances that did not react with the analyte follow in the back of the channel. With proper labeling, either before or after the reaction, the section of the channel with the reaction can be identified and its length measured. The longer the length of the microfluidic channel with the reaction in it, the higher the quantity of the analyte in the test sample.

In the present invention the length of the reacted microfluidic channel represents that part of the microfluidic channel where the analyte reacted with the immobilized substances. The length of reacted microfluidic channel where the reaction takes place starts from the reaction beginning point in the microfluidic channel.

The immobilized substances are capable of combining with a solid support, which may be either a portion of the surface of the microfluidic channel or attached to the surface of the microfluidic channel. For example, the solid support is partially or entirely modified with specific functional group. The solid support attached to the surface of the microfluidic channel may be selected from nitrocellulose, latex, nylon, polystyrene or the combination thereof. Another example of a solid support attached to the surface of the microfluidic channel may be beads, particles, magnetic particles, glass fiber or the combination thereof. The solid support attached to the surface of the microfluidic channel may also be a layer of porous materials. One example of immobilized substances and solid support might be antibodies bound to at least a portion of the microfluidic channel with the specific functional groups. Another example of immobilized substances and solid support might be an antibody attached to porous materials inside the walls of the microfluidic channel.

The analyte detection region of the present invention is comprised of at least one type of immobilized substance in the test sample to detect at least one type of analyte. For example, each of the microfluidic channels is provided with one reaction beginning point and placed with one type of immobilized substances. For example, two microfluidic channels are provided with two reaction beginning points and placed with either the same or different types of immobilized substances to compare the quantities of the same analyte or to measure the quantities of two types of analytes.

The microfluidic channel may be linear or curved. In the preferred mode of the present invention, the microfluidic channel is curved. A variety of shapes of a curved microfluidic channel may be used including, but are not limited to, spiral, serpentine, zigzag, arc shaped and the like. The curved shape of the microfluidic channel extends the length of the analyte detection region without requiring a longer chip. The cross-sectional dimension of the channel may be square, rectangular, semicircular, circular, etc.

The analyte detection region may be constructed with a plurality of microfluidic channels, in parallel, in series, or the combination thereof.

In the preset invention, the fluid driving device may be an active fluid driving device, a passive fluid driving device or the combination thereof. An active fluid driving device is a powered device for controlling the speed of the flow of the sample through the microfluidic channel. The active fluid driving device is coupled to at least one portion of the analyte detection region and is capable of varying the speed of the flow over time so that the analyte reacts sequentially with the nonreactive immobilized substances in the microfluidic channel. In one example, the active fluid driving device is a pump. The pump may be an on-chip pump, such as the micro pump made by photolithography process or an off-chip pump. The type of pump may be a syringe pump, a peristaltic pump, or a mechanism that can contract the gas in the channel, pushing it forward by electrical power, mechanical power, a manual operation, a chemical reaction causing gas consumption, the physical change of the chamber volume, low pressure or high-pressure chamber hook-up, etc.

In the present invention, the passive fluid driving device is capable of generating a capillary effect to drive the fluid flow in the microfluidic channel of the analyte detection region. For example, the hydrophilic/hydrophobic property of the microfluidic channel materials may control the auto forward speed so that the analyte has the chance to react sequentially with the nonreactive immobilized substances lined up in microfluidic channel. For example, the forward speed of the sample in a microfluidic channel made of the most hydrophilic material would be faster than that made of a less hydrophilic material.

The microfluidic channel of the analyte detection region further comprises a passive fluid modulating member capable of adjusting the speed of the flow and/or disturbing the fluid in the microfluidic channel to increase the opportunity for contact between the analyte and the immobilized substances. Passive modulating members include, but are not limited to, local modification of the dimensions or shapes of the microfluidic channel; partial or entire inner surface modification of a section of the microfluidic channel using materials selected from hydrophilic materials, hydrophobic materials or a combination thereof; provided with protrusions or depressions at the inner surface of the microfluidic channel. Passive fluid modulating members may be used with an active or passive fluid driving device.

The materials of the sample assay structure for the present invention are either hydrophilic or hydrophobic. One type of structure that may be used with the present invention is constructed of upper and lower substrates. Another is constructed of a substrate and an adhesive tape. Another, of two or more substrates.

The sample assay structure of the microfluidic chip for quantitative analysis may further comprise a pre-treatment mechanism located between the sample inlet port and the analyte detection region to support the modulation of the sample entered into the analyte detection region. A pre-treatment mechanism may involve a mechanism to label the analyte in the test sample. Analyte labeling methods include, but are not limited to: enzymes, fluorescence, luminescence, nano particles or other substances capable of present displaying colors for labeling. Another pre-treatment mechanism may involve a volume control mechanism for modulating the volume of the test sample entering the analyte detection region or a mechanism for modulating the concentration of the test sample entering the analyte detection region. For example, pre-treatment procedures may include diluting or concentrating the sample, or a sample ingredients modulating area, eliminating or adding other ingredients to the sample, such as, removing blood cells in a whole blood sample or a mixing member for mixing ingredients in the sample or a degassing member for excluding air bubbles from the sample.

The sample assay structure of the present invention may further comprise a post-treatment mechanism connected to at least one portion of the analyte detection region to provide for or improve the ability to differentiate reacted vs. nonreacted immobilized substances. A post-treatment mechanism might be a labeling mechanism for providing materials to label the substances that reacted, but remained in the channel following the reaction. Another post-treatment mechanism might be a mechanism to wash the analyte detection region after the reaction to enhance the signal reading results.

In the preferred mode of the present invention, the analyte detection region further includes at least one labeled scale for defining or calculating the quantity of the analyte in the test sample.

The present invention provides a method for processing the quantitative assay of the targeted analyte in a test sample. It includes: providing a test sample; introducing the test sample into a microfluidic channel entrance, wherein the microfluidic channel is provided with reaction beginning point which is started with placing a plurality of immobilized substances thereat, the immobilized substances are capable of reacting with the analyte; controlling the flow speed of the testing sample in the microfluidic channel, the length of reacted microfluidic channel reflects the analyte quantity after the analyte reacting with the immobilized substances.

The preferred mode of the present invention further includes a method of labeling the analyte: enzymes, fluorescence, luminescence, nano-particles or other substances capable of displaying colors.

The preferred mode of the present invention of the structure of the sample assay structure in the microfluidic chip for quantitative analysis includes: a sample inlet port to input a test sample; an analyte detection region, coupled to the sample inlet port, consisting of at least one curved microfluidic channel, in which a plurality of immobilized substances capable of reacting with the analyte are placed; and an active fluid driving device, capable of controlling the speed of the flow of the test sample through the analyte detection region, allowing the quantity of the analyte be indicated by the length of the portion of the microfluidic channel where the analyte reacted with the immobilized substances.

The preferred mode of the present invention includes in the structure a volume control mechanism located between the sample inlet port and the analyte detection region to modulate the volume of the test sample entering the analyte detection region. The preferred mode of the present invention uses a pump as the active fluid driving device. The preferred mode of the present invention involves a passive fluid modulating member in the microfluidic channel of the analyte detection region.

Other objects, advantages, and novel features of the invention will become apparent from the following detailed description as well as from the accompanying drawings.

### BRIEF DESCRIPTION OF THE DRAWINGS

FIG. 1 is a typical strip and the enlarged figure of a part of the image.

FIG. 2 is a practice mode of the quantitative analysis of an analyte using the microfluidic chip of the present invention, using an active fluid driving device.

FIG. 3a is the quantitative analysis of an analyte using the microfluidic chip of the present invention, showing the relationship of flowing speed vs. time of the active fluid driving device with fluids driven at the same speed.

FIG. 3b (A)˜(D) is the quantitative analysis of an analyte using the microfluidic chip of the present invention, showing the relationship of flowing speed vs. time of the active fluid driving device with fluids driven at a variety of speeds.

FIG. 4a is a practice mode of the quantitative analysis of an analyte using the microfluidic chip of the present invention, with the pluralities of immobilized substances placed non-continuously.

FIG. 4b is another practice mode of the quantitative analysis of an analyte using the microfluidic chip of the present invention, with the pluralities of immobilized substances placed non-continuously.

FIG. 5a is a practice mode of the quantitative analysis of an analyte using the microfluidic chip of the present invention, with the pluralities of passive fluid modulating members placed in random locations in the microfluidic channel.

FIG. 5b is a practice mode of the quantitative analysis of an analyte using the microfluidic chip of the present invention, the pluralities of passive fluid modulating members being formed by the special substances in partial or entire modulating microfluidic channel.

FIG. 5c is a practice mode of a fluid modulating member formed by local modification to the dimensions or shapes of said microfluidic channel.

FIG. 5d is a practice mode of a fluid modulating member formed by providing with protrusions or depressions at the inner surface of the microfluidic channel.

FIG. 6a is an example of the labeling scale of the microfluidic channel in the quantitative analysis of an analyte using the microfluidic chip of the present invention.

FIG. 6b is another example of the labeling scale of the microfluidic channel in the quantitative analysis of an analyte using the microfluidic chip of the present invention.

FIG. 7 is an example of custom-designing the dimension of the microfluidic channel to enhance the accuracy of the reading in the quantitative analysis of an analyte using the microfluidic chip of the present invention.

FIG. 8 is a practice mode of the analyte detection region constructed with a plurality of microfluidic channels connected serially in the quantitative analysis of an analyte using the microfluidic chip of the present invention.

FIG. 9 is a practice mode of the analyte detection region constructed with a plurality of microfluidic channels connected serially and in parallel in the quantitative analysis of an analyte using the microfluidic chip of the present invention.

FIG. 10a is a practice mode of the quantitative analysis of an analyte using the microfluidic chip of the present invention with a pretreatment mechanism connected to it.

FIG. 10b is a practice mode of the quantitative analysis of an analyte using the microfluidic chip of the present invention with a post treatment mechanism connected to it.

FIG. 10c is a practice mode of the quantitative analysis of an analyte using the microfluidic chip of the present invention with both a pretreatment mechanism and a post treatment mechanism connected to it.

FIG. 10d is another practice mode of the quantitative analysis of an analyte using the microfluidic chip of the present invention with both a pretreatment mechanism and a post treatment mechanism connected to it.

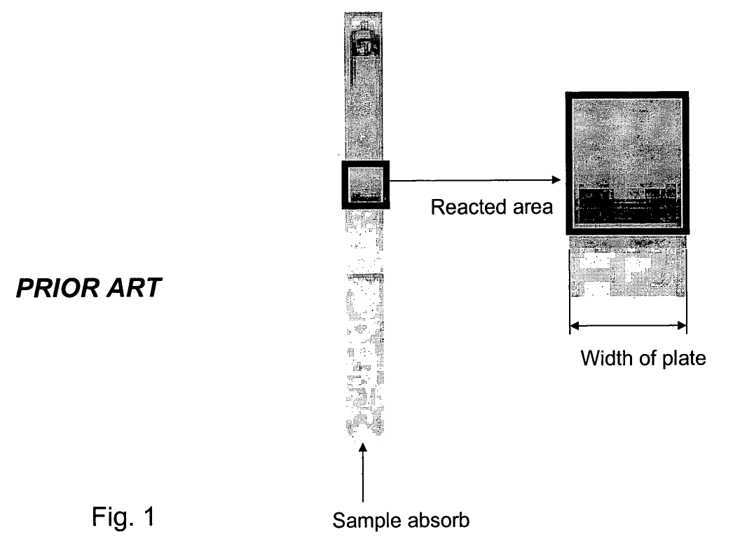

### DETAILED DESCRIPTION OF THE PREFERRED EMBODIMENT

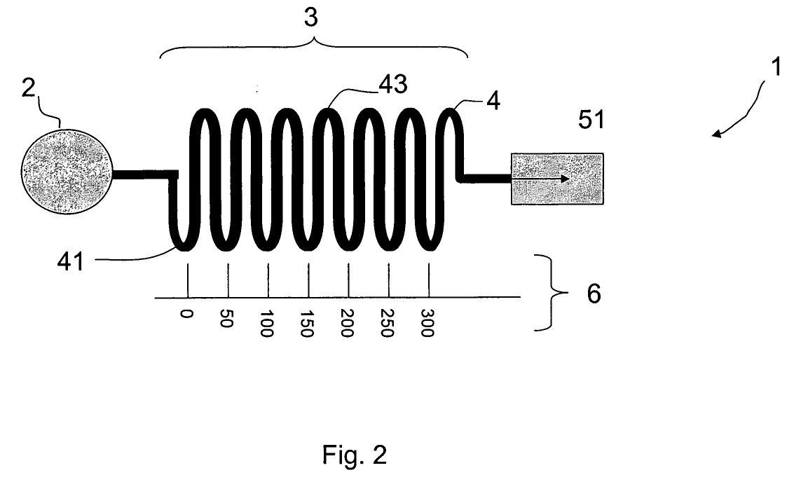

The purpose of the present invention is to develop an analyte assay structure capable of performing quantitative assays without the use of an instrument. As shown in FIG. 2, the structure 1 is comprised of: a sample inlet port 2 for inputting a testing sample; an analyte detection region 3 including at least one microfluidic channel 4 coupled to the sample inlet port 2. The microfluidic channel 4 has a plurality of immobilized substances 43 (shown as horizontal lines in FIG. 2) from the reaction beginning point 41. The immobilized substances are capable of reacting with the analyte. The quantity of the immobilized substances often exceeds the quantity of the analyte that may be needed for the reaction. The immobilized substances are attached to a solid support (for example, the solid support is a portion of the microfluidic channel surface) and a fluid driving device capable of controlling the speed of the flow of the test sample in the analyte detection region 3. The analyte reacts with the immobilized substances 43 sequentially from the reaction beginning point 41. The immobilized substances that react concentrate at the front section of the microfluidic channel 4. The length of the microfluidic channel that reacted (the section with solid color in FIG. 2 ), therefore, reflects the quantity of the analyte.

The analyte detection region 3 includes a scale 6 for reading the length of the channel that reacted or the calibrated quantity of the analyte. The scale may be numbers printed on or attached to the analyte detection region 3, or special geometric features of the microfluidic channel designed to represent the levels of the analyte.

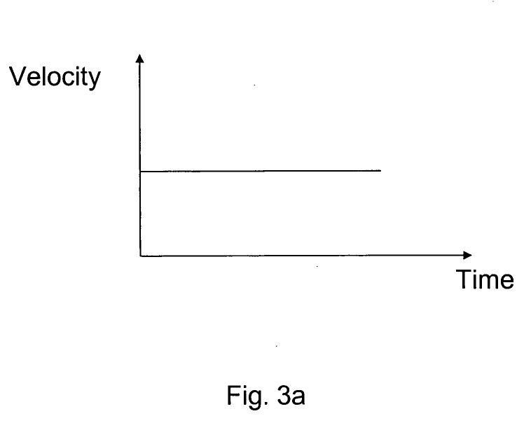

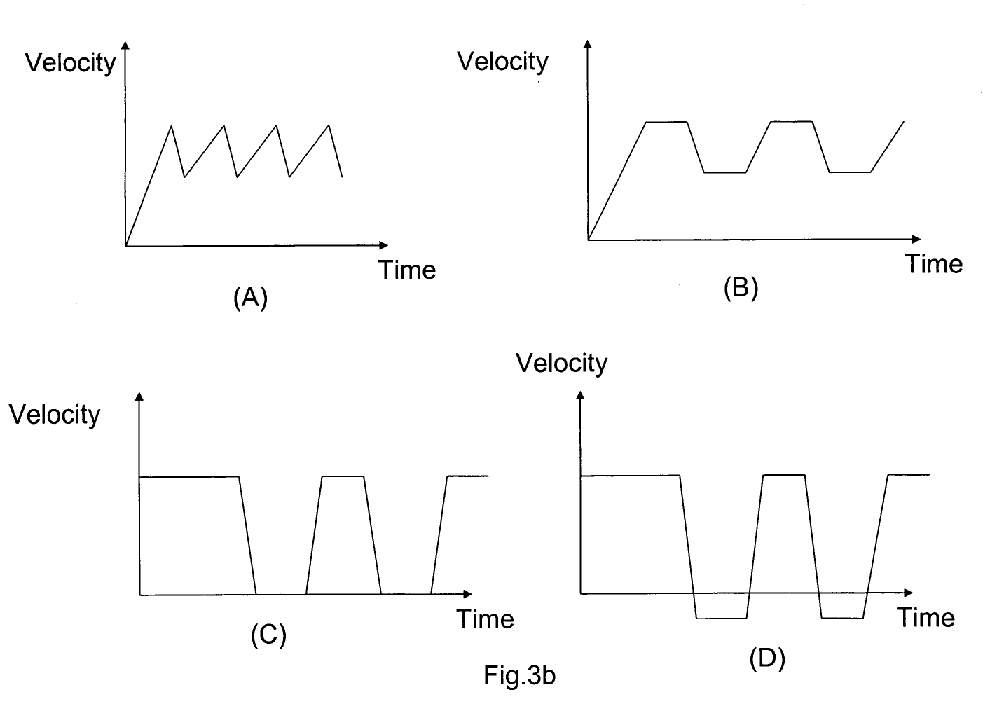

The fluid driving device may be either an active fluid driving device, a passive fluid driving device, or the combination of the two. An active fluid driving device 51 is a powered device capable of driving the sample in the microfluidic channel in a variety of patterns, such as a consistently slow-forward motion (FIG. 3a), an alternating back-and-forth motion (with the forward step larger than the back step so the overall effect is a forward motion), or an alternating stop-and-go motion (FIG. 3b(A)-(D)). By prolonging the sample retention time and/or by generating flow turbulence, sufficient opportunities exist for contact between the analyte and the immobilized substances. Flow patterns as such allow for sequential reactions with the immobilized substances 43 from the reaction beginning point 41. An active fluid driving device 51 may be a device capable of varying the speed of the fluid flow over time, for example, a pump, coupled to at least one portion of the analyte detection region 3.

In one example of the present invention, a well between the microfluidic channel of the analyte detection region and the active fluid driving device is provided for collecting liquid waste. The pump is turned off after the test sample completes its reaction and flows into the waste collection well.

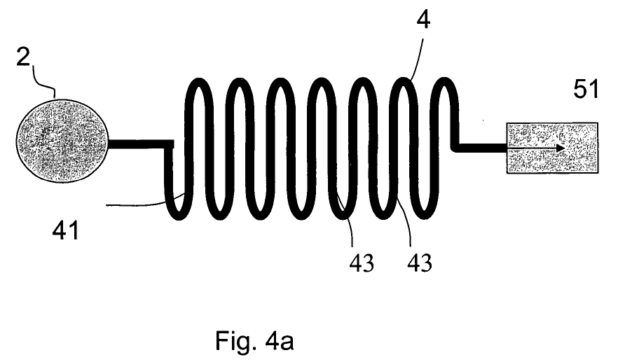

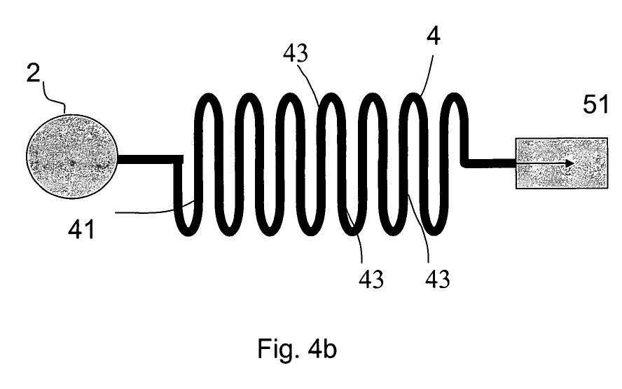

In another example of the present invention, a plurality of immobilized substances are placed continuously (as shown in FIG. 2), or intermittently (as shown in FIGS. 4a, 4b) in the microfluidic channel.

In another example of the present invention, the passive fluid-driving device drives the flow by capillary action inside the microfluidic channel.

By the use of materials with the appropriate hydrophilic/hydrophobic properties for the microfluidic channel, the flow speed of the sample can be controlled to allow sequential reaction between the analyte and the immobilized substances. For example, the forward speed of the sample in a microfluidic channel made of the most hydrophilic material is faster than that in a microfluidic channel made of a less hydrophilic material.

In another example of present invention, the sample assay structure is composed of two substrates. One of the substrates has patterns of open channels and wells, and the other substrate is either flat or has patterns too. The bonding of the two substrates forms the sample assay structure, allowing liquid to flow within the channels and wells and carry out all sorts of missions. Another example of the sample assay structure is composed of a substrate and an adhesive tape. Another is constructed of two or more substrates.

The materials of the sample assay structure for the present invention are either hydrophilic or hydrophobic. Structure materials may be selected from, but are not limited to, polydimethylsiloxane (PDMS), polycarbonate (PC), cyclic olefin copolymers (COC), polystyrene (PS), polymethylmethacrylate (PMMA), silicone, PU, PEEK ABS, PP, PET, PTFE, PVDF, POM, UPE, HOPE, PVC, glass, silicon or a combination of two or more of them.

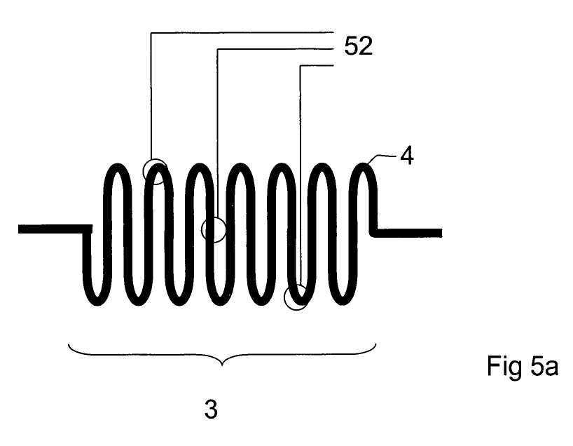

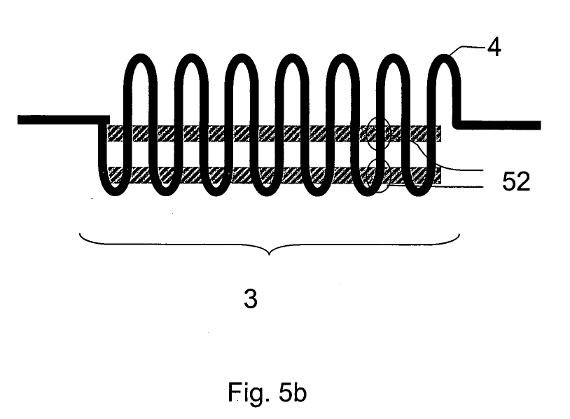

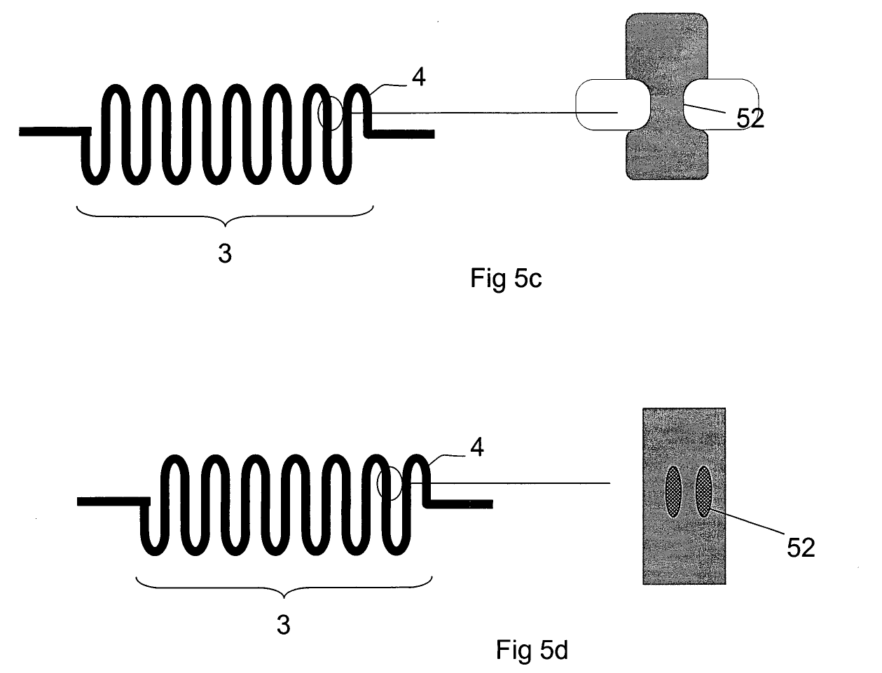

The sample assay structure in microfluidic chip for quantitative analysis of present invention further comprises one or more passive fluid-modulating member capable of adjusting the speed of the flow and/or disturbing the fluid in the microfluidic channel to increase the opportunity for contact between the analyte and the immobilized substances. The multiple fluid modulating members 52 can be placed at any location inside the microfluidic channel, as shown in FIG. 5a. FIG. 5b shows another embodiment of the passive fluid modulating member. The passive fluid-modulating member 52 is formed by modifying the surface inside the microfluidic channel, as the hatched area shown in FIG. 5b. Specific functional groups, hydrophilic or/and hydrophobic materials are possible materials used for surface modification so that local flow speed can be optimally adjusted while the sample enters various sections of the analyte detection region. FIG. 5C shows another embodiment of the passive fluid modulating member 52 in which local modification to the dimensions or shapes of the microfluidic channel is applied to adjust the flow speed or to promote turbulent flow. FIG. 5d shows another embodiment of the passive fluid modulating member 52, in which protrusions or depressions are designed at the inner surface of the microfluidic channel, also for adjusting the flow speed or promoting turbulent flow so the contact opportunity between the analyte and the immobilized substances is increased.

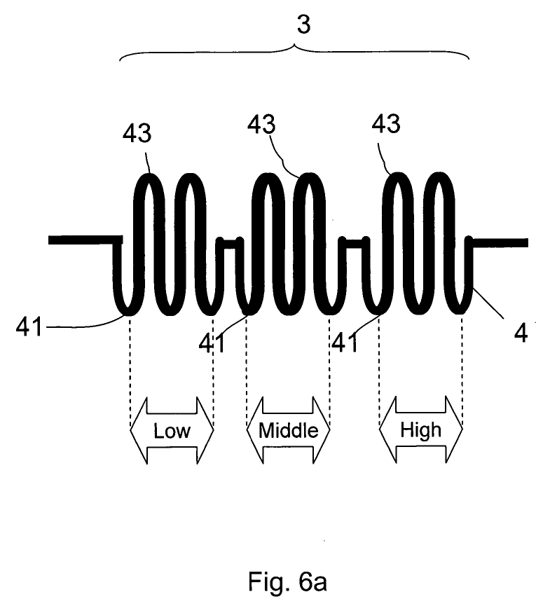

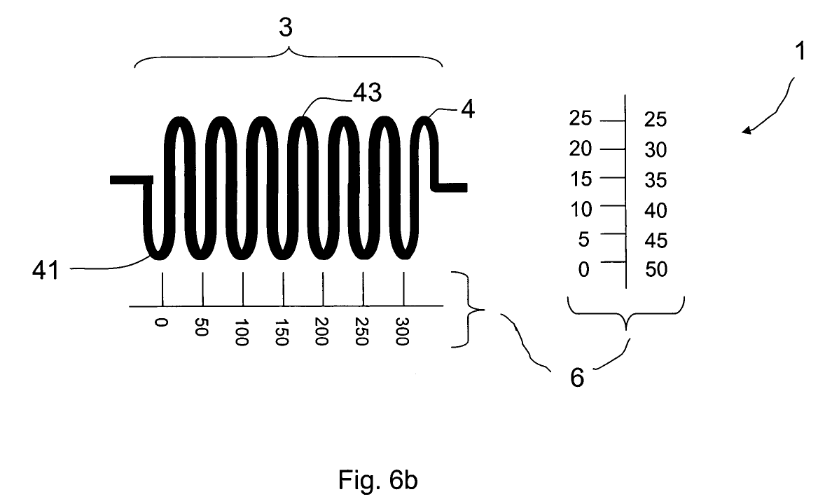

To determine the quantity of the analyte in the sample, the analyte detection region 3 may include a scale 6 to quantify the analyte. The scale may be numbers printed on or attached to the analyte detection region 3, or be special geometric features of the microfluidic channel designed to represent the levels of the analyte. For example, the user may recognize the channel bend location where the channel with reaction ends and relate it to the level of the analyte concentration. FIG. 6a shows an embodiment of the scale 6 near the analyte detection region 3, by which the level of low, medium and high can easily be estimated. FIG. 6b shows an analyte detection region 3 with more bends and a scale with a finer resolution to quantify the analyte. Any geometric shapes or features of the microfluidic channel layout, such as circular, spiral or rectangular, can be used in quantifying the analyte.

The thickness of the microfluidic channels can also be adjusted to enhance the reading resolution, as shown in FIG. 7. A thin channel is preferred when higher resolution in reading is required, while a thick one is preferred when resolution is not an issue but minimizing the area of the chip is. In another embodiment of the quantity reading, the geometric feature and/or the labeled scale can be used alone or together to assist in reading.

The scale may indicate the length of the channel that reacted, then the reading can be converted to the real quantity of the analyte by a look up table or a calibration curve. Or, the scale may indicate directly the calibrated quantity of the analyte without manual conversion.

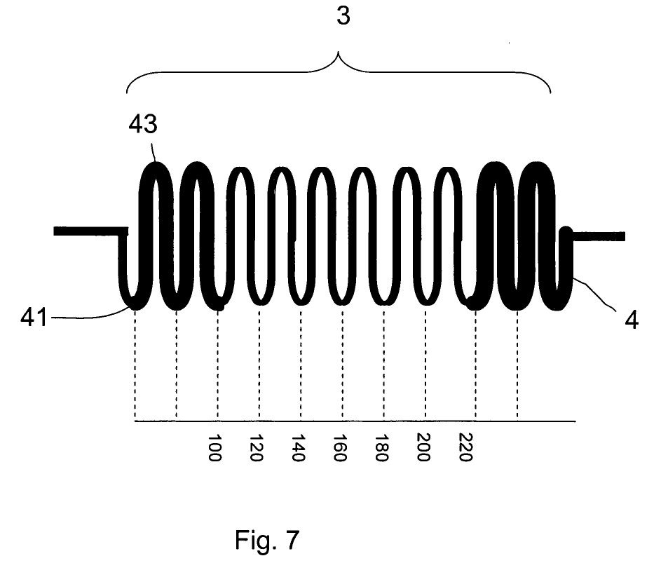

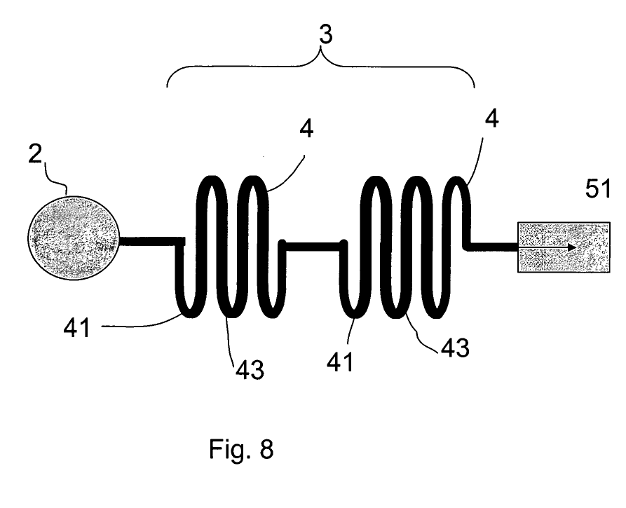

FIG. 8 shows an example of the sample assay structure 1 in microfluidic chip for quantitative analysis of the present invention, which comprises a sample inlet port 2, an analyte detection region 3, which is constructed by a plurality of microfluidic channels, for example, two microfluidic channels 4 constructed in the way of series connection, and an active fluid driving device 51, for example, a pump. One microfluidic channel is provided with one reaction beginning point, starting from which the immobilized substances are placed. One microfluidic channel has one type of the immobilized substances 43 attached. For example, two microfluidic channels are provided with two reaction beginning points and may have the same or different type of immobilized substances attached, for the purposes of multi-analyte assay, comparison, reference, or simply as a control.

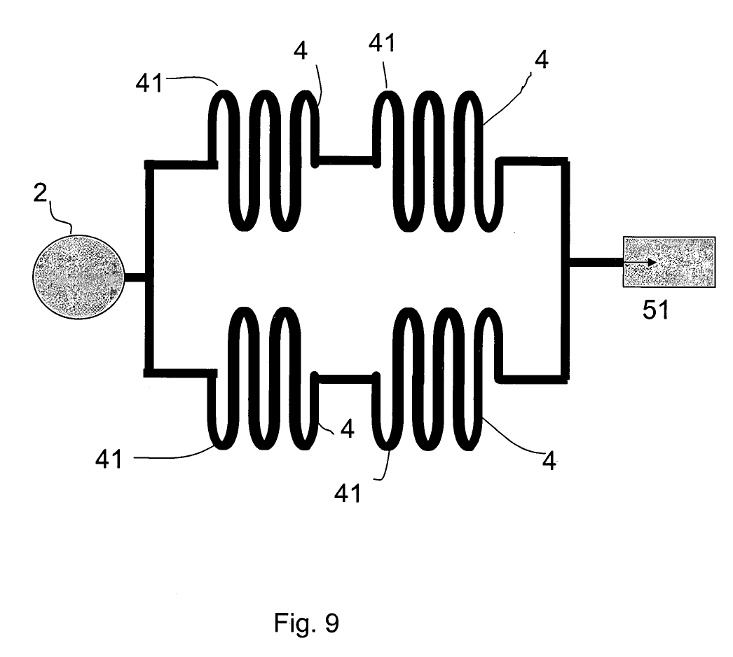

FIG. 9 shows an example of the sample assay structure 1 in microfluidic chip for quantitative analysis of the present invention, which comprises a sample inlet port 2, an analyte detection region 3, which is constructed by a plurality of microfluidic channels, for example, four microfluidic channels 4 constructed in the way of series/parallel connection, and an active fluid driving device 51, for example, a pump. Each microfluidic channel is provided with one reaction beginning point, following that one type of the immobilized substances 43 is attached. In one branch, two microfluidic channels each with its own reaction beginning points are placed with the same or different type of immobilized substances for multiple analyte assay, for comparison, or as a control.

In another embodiment of the sample assay structure for quantitative analysis, the structure comprises multiple sample inlets, at least one analyte detection region and at least one fluid driving device. The same or different types of immobilized substances can be attached to the microfluidic channel inside of the analyte detection region.

In another embodiment of the sample assay structure, the immobilized substances placed in the microfluidic channel can be selected from the list of, but not limited to antibody, antigen, nucleic acid, ligand, receptor, enzymes, peptide or protein or other biological or chemical substances so as to match the type of analyte to be detected.

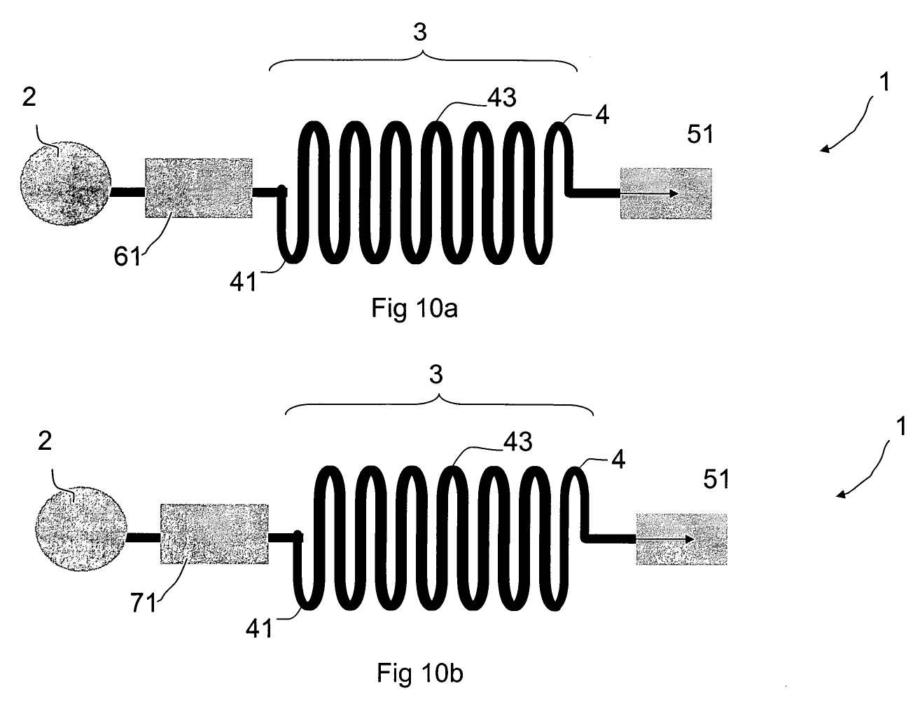

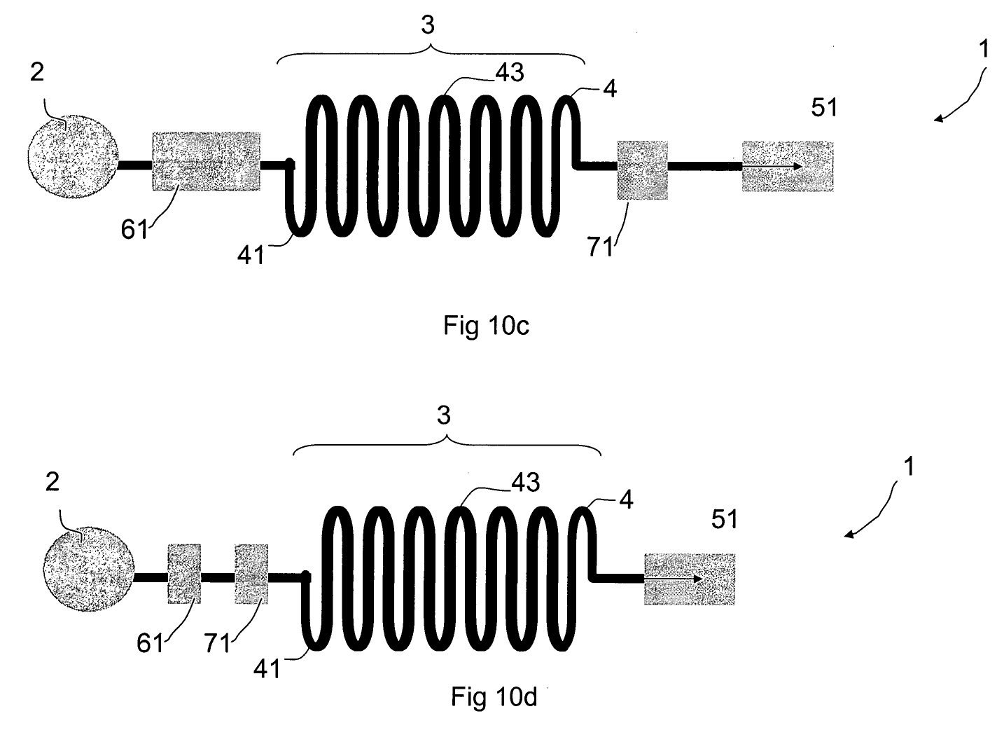

As shown in FIGS. 10a, 10b, 10c, and 10d, the sample assay structure may further comprise a pre-treatment mechanism 61 or a post treatment mechanism 71. For example, a pre-treatment mechanism 61 for modulating the sample condition could be assembled between the sample inlet port 2 and the analyte detection region 3, as displayed in FIG. 10a. The assay structure may further comprise a post treatment mechanism 71 to provide or improve the identification of the analyte in analyte detection region, as shown in FIG. 10b.

The sample assay structure may comprise a combination of pre-treatment mechanism 61 and post treatment mechanism 71, as displayed in FIGS. 10c and 10d. The pre-treatment mechanism 61 may comprise an analyte labeling mechanism for labeling the analyte of the sample. Methods of labeling the analyte include enzyme, fluorescence, luminescence, nano-particles or other substances provided with color effect. For example, the analyte can be conjugated with color particles, or antibody binding color particles. Molecules with dying capability may also be attached to the analyte. The pre-treatment mechanism may also comprise a volume control mechanism to control the volume admitted to the analyte detection region; or a sample concentration modulating mechanism to dilute or concentrate the sample; or a sample composition modulating mechanism to eliminate, add or change the composition of the sample, for example, removing or destroying the blood cells in the blood; or a mixing member to improve uniformity of the solution; or a degassing member to exclude air bubbles in the sample.

The post-treatment mechanism may be a washing mechanism to wash the analyte detection region after reaction.

In another example of the present invention, an instrument may also be used to read the labels of the analyte, whether the labels be in visible light spectrum or not. Then the length of the channel with reactions can be measured and the analyte be quantified. The advantage of applying the present invention together with a reader is that the reader used does not have to be highly sensitive. Since the reacted immobilized substances condense at a compact region, the reading signal of the region is high, the sensitivity requirement of the reading instrument is not as critical as the case when the present invention is not applied.

One preferred mode of the sample assay structure further comprises at least one labeled scale for reading the quantity of the analyte.

The analyte detection region of present invention further comprises a reading module to read length of the microfluidic channel in which the immobilized substances react with the analyte; an analysis module to convert the data into analyte quantity by a look up table or a calibration curve; and a display device to show the assay results.

The present invention provides a method for processing a quantitative assay of the targeted analyte in a testing sample comprising: providing a testing sample; introducing the testing sample into a microfluidic channel entrance, wherein the microfluidic channel is provided with reaction beginning point, which is started with placing a plurality of immobilized substances thereat, the immobilized substances are capable of reacting with the analyte; controlling the flow speed of the testing sample in the microfluidic channel, the length of reacted microfluidic channel reflects the analyte quantity after the analyte reacting with the immobilized substances.

In a preferred mode, the method further comprises a sample labeling process for labeling the analyte in the sample; the method of labeling the analyte may include using enzyme, fluorescence, luminescence, nano-particles or other substances provided with color effect to directly or indirectly affect the analyte, such as conjugating with color particles, antibody binding color particles or attaching molecules with dying capability.

If labels in visible spectrum, such as gold particles, is used, then the sample assay structure or method in the present invention can be applied without the need of a reading instrument.

In a preferred mode, the sample assay structure in microfluidic chip for quantitative analysis of present invention comprises: a sample inlet port for inputting a testing sample; an analyte detection region, which is coupled to the sample inlet port, the analyte detection region comprising at least one curved microfluidic channel, the microfluidic channel is provided with a reaction beginning point following that a plurality of immobilized substances are attached, the immobilized substances are capable of reacting with the analyte; and an active fluid driving device, which is capable of controlling the flow speed of the testing sample in the analyte detection region, the fluid driving device allows the length of reacted microfluidic channel to reflect the analyte quantity after the analyte reacting with_the immobilized substances.

In another preferred mode, the sample assay structure in microfluidic chip for quantitative analysis of present invention further comprises a sample labeling process using labels in visible spectrum, such as gold particles, and a scale near the analyte detection region, then the sample assay structure or method in the present invention can be applied without the need of a reading instrument.

In another preferred mode of the present invention, the sample assay structure further comprises a volume control mechanism between the sample inlet port and the analyte detection region for controlling the volume of the testing sample entering the analyte detection region.

One preferred mode of the present invention, wherein the active fluid-driving device is a pump. One preferred mode of the present invention, wherein the microfluidic channel of the analyte detection region further comprises a passive fluid modulating member.

The present invention can be applied to quantitative biochemical assay without using any reading instrument. Features of the invention include:
(1) By using a long and thin microfluidic channel in the analyte detection region, the reading resolution can be dramatically enhanced, therefore is good for quantitive, semi-quantitive, and qualitative applications.
(2) The use of a fluid driving device and a passive fluid modulating member promotes sequential reactions and concentrates the reacted sites, thus enhancing the accuracy of quantifying the analyte.
(3) When labels in visible spectrum is used, the sample assay structure or method in the present invention can be applied without the need of a reading instrument.
(4) Pre-treatment and post-treatment mechanisms can be incorporated into the sample assay structure so that a number of steps in a complete assay protocol can be automatically executed with improved assay accuracy while the need of a professional is eliminated.

The following examples are used to further demonstrate the advantages of the present invention and to expand rather than limit its scope.

### EXAMPLE 1

### Applying Speed Control to a Quantitative Assay of the Analyte

In this example photolithoghaphy of MEMS process and polymer materials were applied to make the sample assay structure. Two samples with different quantities of the analyte were tested using the quantitative analyte assay structure presented in the present invention. The structure was constructed of an upper and a lower substrate. The upper substrate was made of hydrophobic polymer on which a serpentine thin channel functions as the sample path and two wells at its two ends function as the sample inlet port and the waste well. The lower substrate was made of glass on which chemical function groups were grafted. The assembly of the two substrates formed the sample assay structure. An external pump was hooked up to the waste well to drive the sample in the microfluidic channel 100 μm in both width and depth. The layout of the chip is similar to the one shown in FIG. 2, each straight section of the U-shaped channel (L) being 8 mm long. EDTA powder, the anticoagulant agent was placed loosely at the sample inlet port. From the reaction beginning point 41, Goat-anti-mouse-IgG, 5.6 mg/ml was immobilized to capture the analyte Mouse-IgG C mixed in whole blood. The analyte was labeled with colloidal gold to display a visible pink color. A sample flow speed of 0.8 mm/min was applied in this case to allow sufficient reaction. Two chips alike were used to test two samples: Chip 1 and chip 2 received the same concentration of the sample Mouse-IgG CGC, OD540=50, but with different volumes—1.5 μl and 3 μl, respectively. The sample was loaded into the inlet well, driven by the pump, and reacted with the immobilized substances on the wall of the channel. Results showed that, with the proper flow speed control, the immobilized substances that reacted were located mostly at the front section of the channel while the back section of the channel remains unchanged in color because the analytes were consumed at the front section. By observing the length of the pink section that reacted the quantity of the analyte can be calibrated.

After reaction the length of the channel that reacted was 88 mm+/−4 mm on chip 1 and 160 mm+/−8 mm on chip 2. The results indicated that the quantity of analyte in chip 1 is half of that of chip 2, as was expected. The results showed that driving the sample at the proper speed through a thin, long and curved microfluidic channel permits the sequential reaction of immobilized substances, allowing quantitative measurement of the analyte based on the measurement of the length of the channel that reacted. No instrument was needed to read the results in this example.

### EXAMPLE 2

### Applying Speed Control and Local Dimension Modifications to a Quantitative Assay of the Analyte

In this example the materials used and the manufacturing process applied to the assay structure were the same as those in Example 1. However, this example differs from the preceding one in that there were local modifications to the dimensions of the channel—a type of passive fluid modulating members was introduced in the present case. In the analyte detection region the 300 um wide channel was reduced in width unsymmetrically to 150 um at every 2 mm distance.

A sample assay structure in the microfluidic chip in this case modulated the reaction by controlling flow speed and by variations in the geometric shapes of the microfluidic channel for quantitative analyte analysis. Goat-anti-mouse-IgG, 5.6 mg/ml was immobilized on the wall of the channel. An active external pump is used to drive the sample making it flow at 12 mm/min, faster than that in Example 1. Two different samples were tested on two chips of the same kind. Chip 1 had 4 ul of the labeled analyte Mouse-IgG CGC, OD540=50 while Chip 2 had 2 ul of the labeled analyte Mouse-IgG CGC, OD540=50 diluted in 2 ul of water. The testing procedure was the same as in Example 1. The length of the pink channel that reacted for chip 1 was 136 mm+/−20 mm. For chip 2 it was 72 mm+/−16 mm. The ratio of the lengths of the two chips corresponded to the ratio of the amounts of analyte loaded into the chips.

### EXAMPLE 3

### Applying Speed Control and Modifications to Local Dimension and Shape to a Quantitative Assay of the Analyte

In this example the materials used, the manufacturing process applied, and the alternating width of the channel (300 um full width narrowed unsymmetrically to 150 um) in the assay structure were the same as those in Example 2. The flow was driven at 12 mm/min as in Example 2.

However, in this example protrusions were located in the analyte detection region so local turbulent flow enhanced the opportunity for contact between the analyte and the immobilized substances. Goat-anti-mouse-IgC, 5.6 mg/ml was immobilized on the wall of the channel.

An active external pump was used to drive the sample making it flow at 12 mm/min, faster than in Example 1. Two different samples were tested on two chips of the same kind. Chip 1 had 6 ul of labeled analyte Mouse-IgG CGC, OD540=50, while Chip 2 had 3 ul of labeled analyte Mouse-IgG CGC, OD540=50 diluted in 3 ul of water, half the concentration of that in chip 1. The testing procedure was the same as in Example 2. The length of the pink channel that reacted for chip 1 was 112 mm+/−8 mm. The length of the pink channel that reacted for chip 2 was 64 mm+/−8 mm. The ratio of the lengths of the two chips corresponded to the ratio of the amounts of analyte loaded into the chips.

Conclusions can be drawn from these examples indicating that the sample assay structure in the present invention allows the quantitative measurement of analyte in the sample. No reading instrument was used in any of these three examples.

Although the present invention is demonstrated in relation to the preferred embodiment, it is to be understood that many other possible modifications and variations may be made without departing from the spirit and scope of the invention as claimed herein.

What is claimed is:

1. A sample assay structure in microfluidic chip for quantitative analysis comprising:
a sample inlet port for inputting a testing sample;
an analyte detection region, coupled to said sample inlet port, consisting of at least one microfluidic channel, in which a plurality of immobilized substances capable of reacting with said analyte are placed; and
a fluid driving device, capable of controlling the speed of the flow of said test sample through said analyte detection region, allowing the quantity of said analyte be indicated by the length of the portion of the microfluidic channel where said analyte reacted with said immobilized substances.

2. The sample assay structure of claim 1, wherein said microfluidic channel of said analyte detection region is in curved shape.

3. The analyte assay structure of claim 1, wherein said fluid driving device is selected from an active fluid driving device, a passive fluid driving device or the combination thereof.

4. The sample assay structure of claim 3, wherein said active fluid driving device is coupled to at least one portion of said analyte detection region.

5. The sample assay structure of claim 3, wherein said active fluid driving device is capable of varying the speed of fluid flow over time.

6. The sample assay structure of claim 3, wherein said active fluid driving device is a pump.

7. The sample assay structure of claim 3, wherein said passive fluid driving device is capable of generating a capillary effect to drive fluid flow in said microfluidic channel of said analyte detection region.

8. The sample assay structure of claim 1, wherein said microfluidic channel of said analyte detection region further comprises a passive fluid modulating member.

9. The sample assay structure of claim 8, wherein said modulating member of said analyte detection region comprises local modification to the dimensions or shapes of said microfluidic channel.

10. The sample assay structure of claim 8, wherein said modulating member of said analyte detection region is a portion of said microfluidic channel and made from hydrophilic materials, hydrophobic materials or the combination thereof to process an entire or partial inner surface modification of said portion in said microfluidic channel.

11. The sample assay structure of claim 8, wherein said passive fluid modulating member is provided with protrusions or depressions at the inner surface of said microfluidic channel.

12. The sample assay structure of claim 1, wherein said structure materials are either hydrophilic or hydrophobic.

13. The sample assay structure of claim 1, wherein said structure is composed of upper and lower substrates

14. The sample assay structure of claim 1, wherein said structure is composed of a substrate and an adhesive tape.

15. The sample assay structure of claim 12, wherein said structure materials are selected from polydimethylsiloxane (PDMS), polycarbonate (PC), cyclic olefin copolymers (COC), polystyrene (PS), polymethylmethacrylate (PMMA), silicone, PU, PEEK ABS, PP, PET, PTFE, PVDF, POM, UPE, HOPE, PVC, glass, silicon or the combination thereof.

16. The sample assay structure of claim 1, wherein said immobilized substances comprises one of antibody, antigen, nucleic acid, ligand, receptor, enzymes, peptide and protein.

17. The sample assay structure of claim 1, wherein said immobilized substances are coupled to a solid support, and said solid support is a portion of the surface of said microfluidic channel.

18. The sample assay structure of claim 17, wherein said solid support is either partially or entirely modified by a specific functional group.

19. The sample assay structure of claim 1, wherein said immobilized substances are coupled to a solid support that is attached to the surface of said microfluidic channel.

20. The sample assay structure of claim 19, wherein said solid support is either partially or entirely modified by a specific functional group.

21. The sample assay structure of claim 19, wherein said solid support is selected from nitrocellulose, latex, nylon, polystyrene or the combination thereof.

22. The sample assay structure of claim 19, wherein said solid support is selected from beads, particles, magnetic particles, glass fiber or the combination thereof.

23. The sample assay structure of claim 19, wherein said solid support is a layer of porous materials.

24. The sample assay structure of claim 1, wherein said analyte detection region is constructed of a plurality of microfluidic channels connected in parallel or as a series or the combination thereof.

25. The sample assay structure of claim 24, wherein said a plurality of microfluidic channels are placed with at least one type of immobilized substances to detect at least one type of analyte.

26. The sample assay structure of claim 1, wherein said at least one microfluidic channel provided with at least one reaction beginning point.

27. The sample assay structure of claim 1, wherein said structure further comprises a pre-treatment mechanism located between said sample inlet port and said analyte detection region to support the modulation of the sample entered into said analyte detection region.

28. The sample assay structure of claim 27, wherein said pre-treatment mechanism further comprises a sample labeling mechanism to label said analyte.

29. The sample assay structure of claim 27, wherein said pre-treatment mechanism further comprises a volume control mechanism to modulate the volume of said testing sample entered into said analyte detection region.

30. The sample assay structure of claim 27, wherein said pre-treatment mechanism further comprises a sample concentration modulating mechanism for modulating the concentration of said testing sample entered said analyte detection region.

31. The sample assay structure of claim 27, wherein said pre-treatment mechanism further comprises a sample composition modulating mechanism to eliminate or add other ingredients to said sample.

32. The sample assay structure of claim 27, wherein said pre-treatment mechanism further comprises a degassing member to exclude air bubbles from said sample.

33. The sample assay structure of claim 1, wherein said structure further comprises a post-treatment mechanism coupled to at least one portion of said analyte detection region to provide or improve the identification of said analyte in said analyte detection region.

34. The sample assay structure of claim 33, wherein said post-treatment mechanism further comprises a washing mechanism to wash said analyte detection region after the reaction.

35. The sample assay structure of claim 1, wherein said analyte detection region further comprises at least one labeled scale to define or calculate the quantity of said analyte.

36. A method for processing a quantitative assay of a targeted analyte in a testing sample comprising:
providing a testing sample;
introducing said testing sample into the entrance of a microfluidic channel, wherein said microfluidic channel is provided with reaction beginning point which is started with placing a plurality of immobilized substances thereat, said immobilized substances are capable of reacting with said analyte;
controlling the speed of the flow of said test sample in said microfluidic channel, the length of reacted microfluidic channel reflecting the quantity of said analyte after said analyte reacting with said immobilized substances.

37. The method of claim 36, wherein said method further comprises a sample labeling process.

38. A sample assay structure in microfluidic chip for quantitative analysis comprising:
a sample inlet port for inputting a testing sample;
an analyte detection region, coupled to said sample inlet port, consisting of at least one curved microfluidic channel, in which a plurality of immobilized substances capable of reacting with said analyte are placed; and
an active fluid driving device, capable of controlling the speed of the flow of said test sample through said analyte detection region, allowing the quantity of said analyte be indicated by the length of the portion of the microfluidic channel where said analyte reacted with said immobilized substances.

39. The sample assay structure of claim 38, wherein said structure further comprises a volume control mechanism located between said sample inlet port and said analyte detection region to modulate the volume of said test sample entered into said analyte detection region.

40. The sample assay structure of claim 38, wherein said structure further comprises a sample labeling mechanism located between said sample inlet port and said analyte detection region to label said analyte.

41. The sample assay structure of claim 38, wherein said active fluid driving device is a pump.

42. The sample assay structure of claim 38, wherein said microfluidic channel of said analyte detection region further comprises a passive fluid modulating member.
Reference:
[1] Se-Hwan Paek, et. al., Development of Rapid One-Step Immunochromatographic Assay, Methods 22, p.53-p.60, 2000
